(19) United States
(12) Patent Application Publication
(10) Pub. No.: US 2012/0276552 A1
(43) Pub. Date: Nov. 1, 2012

(54) SERUM SPLA2-IIA AS DIAGNOSIS MARKER FOR PROSTATE AND LUNG CANCER

(75) Inventor: Shan Lu, Cincinnati, OH (US)

(73) Assignee: UNIVERSITY OF CINCINNATI, Cincinnati, OH (US)

(21) Appl. No.: 13/520,586

(22) PCT Filed: Jan. 5, 2011
(86) PCT No.: PCT/US2011/020225

§ 371 Date: Jul. 5, 2012

Related U.S. Application Data
(60) Provisional application No. 61/292,270, filed on Jan. 5, 2010.
(60) Provisional application No. 61/400,606, filed on Jul. 30, 2010.
(60) Provisional application No. 61/400,806, filed on Aug. 3, 2010.

(51) Int. Cl. G01N 33/574; C12Q 1/44

(52) U.S. Cl. 435/7.4; 435/19

(57) ABSTRACT

Methods for diagnosing prostate cancer and lung cancer are disclosed. The methods include obtaining a biological sample from a subject, determining a level of serum secretory phospholipase A₂-IIA in the biological sample, comparing the level of serum secretory phospholipase A₂-IIA with a baseline level of serum secretory phospholipase A₂-IIA, and diagnosing prostate cancer or lung cancer in the subject. An elevated level of serum secretory phospholipase A₂-IIA as compared to the baseline level correlates to a positive diagnosis of prostate cancer or lung cancer in the subject.

This application claims priority under 35 U.S.C. §119 to U.S. Provisional Application Ser. No. 61/292,270, filed Jan. 5, 2010, U.S. Provisional Application Ser. No. 61/400,606, filed Jul. 30, 2010, and U.S. Provisional Application Ser. No. 61/400,806, filed Aug. 3, 2010, the contents of which are hereby incorporated by reference in their entirety.

The present disclosure relates to methods for diagnosing cancer. More specifically, the present disclosure relates to methods for diagnosing prostate cancer and lung cancer by determining the level of serum secretory phospholipase A₂-IIA.

It is widely accepted that many cancers arise from chronic inflammation. Chronic inflammation is a pathological condition characterized by concurrent active inflammation, tissue destruction, and attempted repair. Chronic inflammation results in a sustained innate immune response which creates a microenvironment rich in cytokines, chemokines, growth factors, and angiogenesis factors, and fosters cell proliferation and survival, a critical step in carcinogenesis. The nuclear factor-κB (hereinafter “NF-κB”) is a key linking molecule in inflammation and immunity to cancer development and progression. The NF-κB target genes, such as cyclooxygenase-2 (hereinafter “COX2”), matrix metalloproteinase (hereinafter “MMP”), VEGF, IL6, and IL8, also play a critical role in cell proliferation, angiogenesis, metastasis, and inflammation. Various carcinogens, oncogenes, and cell signaling pathways, such as EGFR-HER2-PI3K-Akt, activate NF-κB. Activation of NF-κB leads to expression of inflammatory cytokines and growth factors, blockade of apoptosis, promotion of proliferation, angiogenesis, and tumor invasion.

Prostate cancer and benign prostatic hyperplasia (hereinafter “BPH”) are two common male urinary diseases, which are often associated with overlapping signs and symptoms. BPH, a treatable disease, is a nonmalignant enlargement of the prostate; in contrast, cancer of the prostate is the second leading cause of cancer death among men in the United States. Standard diagnostic tests for prostate cancer include prostate specific antigen (hereinafter “PSA”), histopathology, Gleason score, and magnetic resonance imaging (hereinafter “MRI”). However, these diagnostic tests are limited; for example, PSA tests lack sensitivity and specificity and have not been validated in prostate cancer surveillance trials, biopsies are prone to sampling errors, repeated biopsies trigger inflammation, and MRI can miss small tumors. Additionally, PSA levels are high in both BPH and prostate cancer. As a result, it is estimated that greater than approximately 500,000 men will be subjected to unnecessary biopsies each year. Accordingly, there remains a need for improved methods for diagnosing prostate cancer.

Lung cancer is the most common cancer worldwide in both incidence and mortality; for example, approximately 1.3 million new cases of lung cancer are diagnosed each year and approximately 1.2 million deaths result from lung cancer each year. In the United States, lung cancer is the leading cause of cancer death. Additionally, lung cancer has a much lower survival rate when compared to other common cancers; this is partly due to the fact that over 50% of patients receive late diagnoses of locally-advanced or metastatic disease. Standard diagnostic tests for lung cancer include low dose spiral CT (hereinafter “LDCT”), chest radiographs (hereinafter “CSR's”), and sputum cytology. While increased sensitivity of imaging technology in LDCT has allowed for the detection of lung cancer at an earlier stage, LCDT is limited in its inability to distinguish malignant nodules from benign tumors and/or inflammatory pseudo tumors. Accordingly, there also remains a need for improved methods for diagnosing lung cancer.

The present disclosure is based on the discovery that serum secretory phospholipase A₂-IIA, (hereinafter “serum sPLA2-IIA”), is a serum diagnosis marker for prostate and/or lung cancer. sPLA2-IIA is both a target and effector gene of NF-κB. Moreover, sPLA2-IIA is a secretory phospholipid hydrolase that mediates the release of arachidonic acid and lysophosphatidylcholine. Accordingly, in one embodiment, a method for diagnosing prostate cancer in a subject is disclosed. The method comprises: (a) obtaining a biological sample from the subject; (b) determining a level of sPLA2-IIA in the biological sample; (c) comparing the level of serum sPLA2-IIA determined in step (b) with a baseline level of serum sPLA2-IIA; and (d) diagnosing prostate cancer in the subject, wherein an elevated level of serum sPLA2-IIA as compared to the baseline level correlates to a positive diagnosis of prostate cancer in the subject.

In another embodiment, a method for diagnosing lung cancer in a subject is disclosed. The method comprises: (a) obtaining a biological sample from the subject; (b) determining a level of serum sPLA2-IIA in the biological sample; (c) comparing the level of serum sPLA2-IIA determined in step (b) with a baseline level of serum sPLA2-IIA; and (d) diagnosing lung cancer in the subject, wherein an elevated level of serum sPLA2-IIA as compared to the baseline level correlates to a positive diagnosis of lung cancer in the subject.

These and other features and advantages of these and other various embodiments according to the present invention will become more apparent in view of the drawings, detailed description, and claims provided herein.

The following detailed description of the embodiments of the present invention can be better understood when read in conjunction with the following drawings, where like structure is indicated with like reference numerals, and in which:

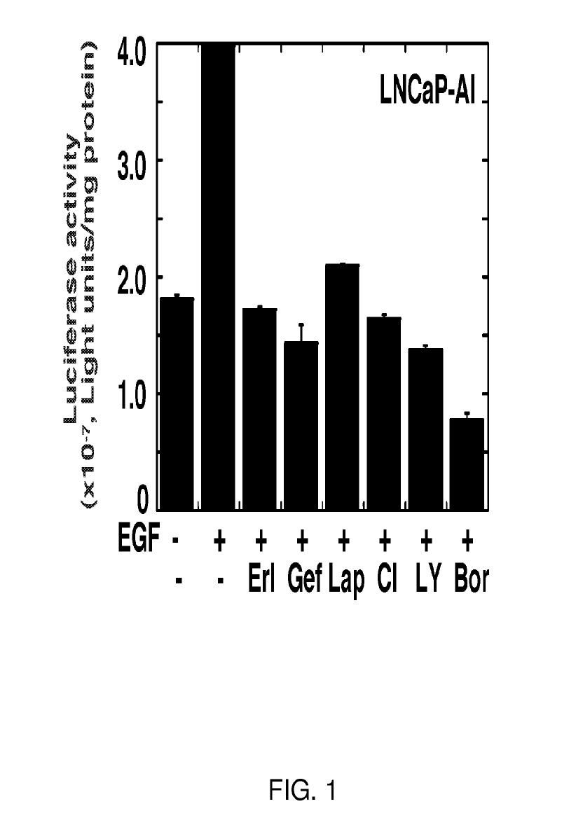

FIG. 1 is a bar graph of serum sPLA2-IIA(-800)-Luc (0.25 μg/well) transfected LNCaP-AI cells (10⁵ cells/well in 12-well plate) and serum sPLA2-IIA(-800)-Luc (0.25 μg/well) transfected LNCap-AI cells (10⁵ cells/well in 12-well plate) treated with epidermal growth factor (100 ng/mL) without or with Erlotinib (˜20 μM), Gefitinib (˜20 μM), Lapatinib (˜20 μM), CI-1033 (˜8 μM), LY294002 (˜20 μM), and Bortezomib (˜20 μM) with respect to luciferase activity (×10⁻⁷, Light units/mg protein);

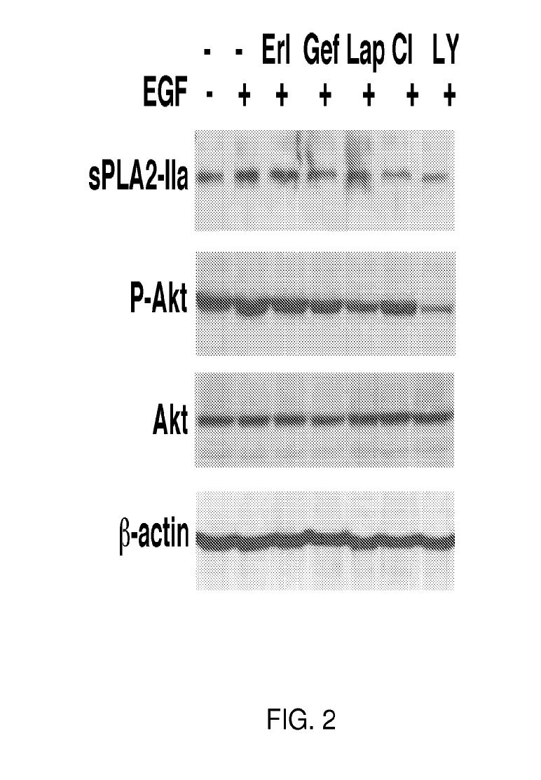

FIG. 2 is a western blot which depicts the expression of serum sPLA2-IIA protein in LNCaP-AI cells treated with Erlotinib (˜20 μM), Gefitinib (˜20 μM), Lapatinib (˜20 μM), CI-1033 (˜8 μM), and LY294002 (˜20 μM) without or with EGF (˜100 ng/mL);

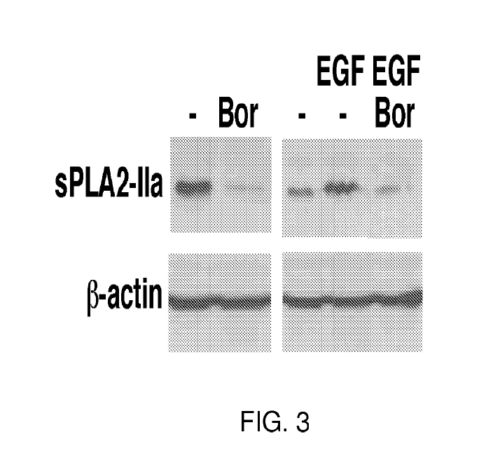

FIG. 3 is a western blot which depicts the expression of serum sPLA2-IIA protein in LNCaP-AI cells treated with Bortezomib (˜20 μM) with or without EGF (˜100 ng/mL);

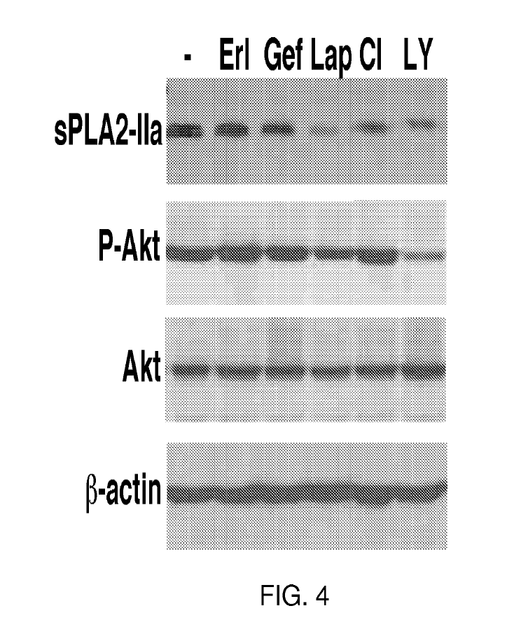

FIG. 4 is a western blot which depicts the expression of serum sPLA2-IIA protein in LNCaP-AI cells treated with Erlotinib (˜20 μM), Gefitinib (˜20 μM), Lapatinib (˜20 μM), CI-1033 (˜8 μM), and LY294002 (˜20 μM);

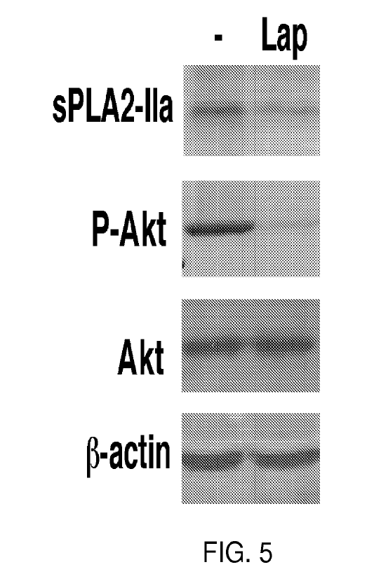

FIG. 5 is a western blot which depicts the expression of serum sPLA2-IIA protein in LNCaP-AI cells treated with Lapatinib (˜20 μM);

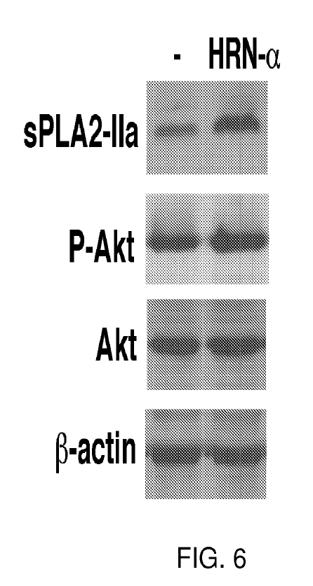

FIG. 6 is a western blot which depicts the expression of serum sPLA2-IIA protein in LNCaP-AI cells treated with Heregulin-α (˜50 ng/mL);

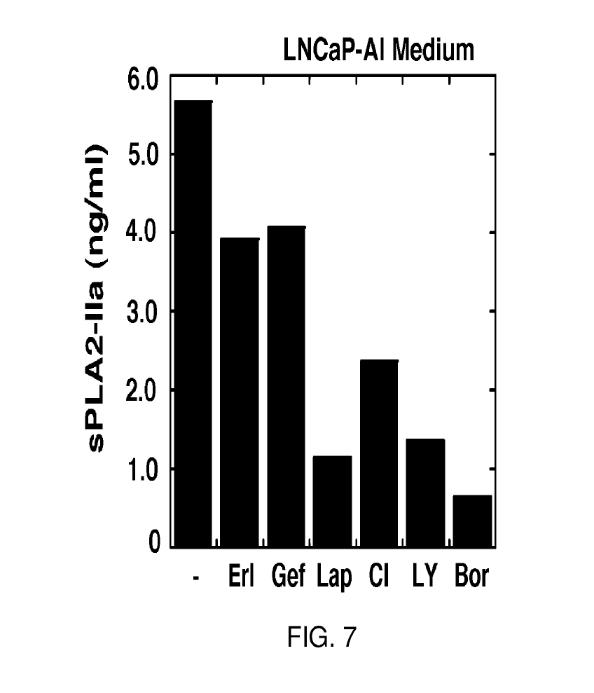

FIG. 7 is a bar graph of serum sPLA2-IIA (ng/mL) in LNCaP-AI cells treated with Erlotinib (˜20 μM), Gefitinib (˜20 μM), Lapatinib (˜20 μM), CI-1033 (˜8 μM), LY294002 (˜20 μM), and Bortezomib (˜20 μM);

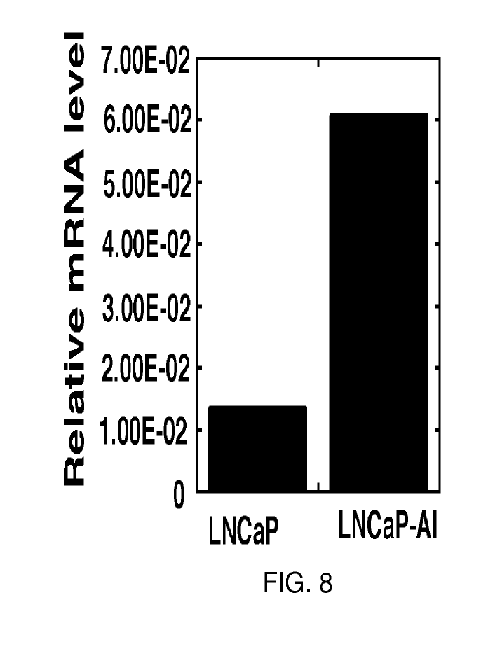

FIG. 8 is a bar graph of mRNA expression levels of serum sPLA2-IIA in LNCaP and LNCaP-AI cells;

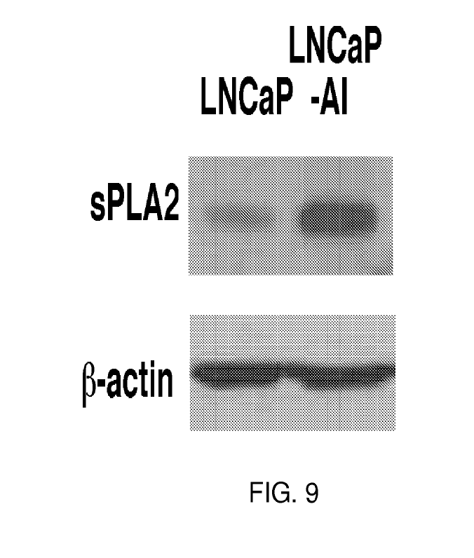

FIG. 9 is a western blot which depicts the expression of serum sPLA2-IIA protein;

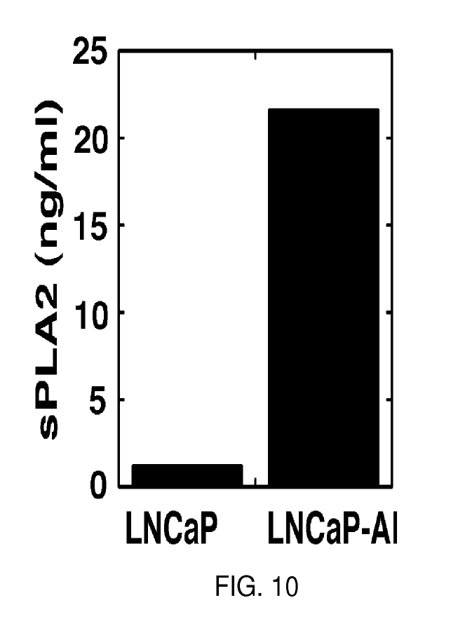

FIG. 10 is a bar graph of sPLA2-IIA (ng/mL) in the conditioned medium secreted by LNCaP-AI (500,000 cells/well in 6 well plate) and LNCaP cells (500,000 cells/well in 6 well plate) by ELISA assay;

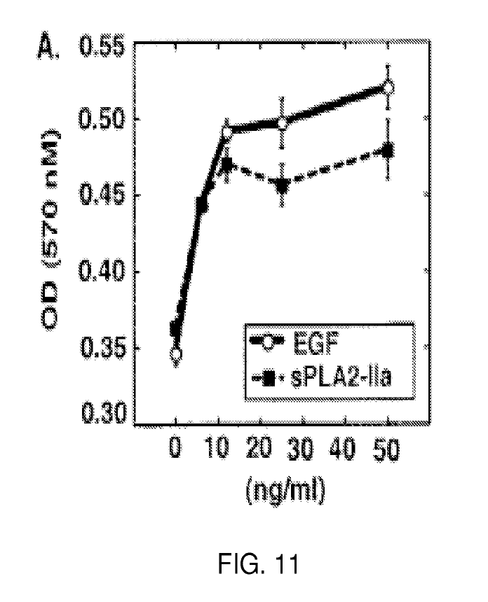

FIG. 11 is a graph of LNCaP-AI cells cultured in 10% stripped medium in the presence of EGF (ng/mL) or serum sPLA2-IIA (ng/mL) for about 4 days with respect to optical density (570 nM);

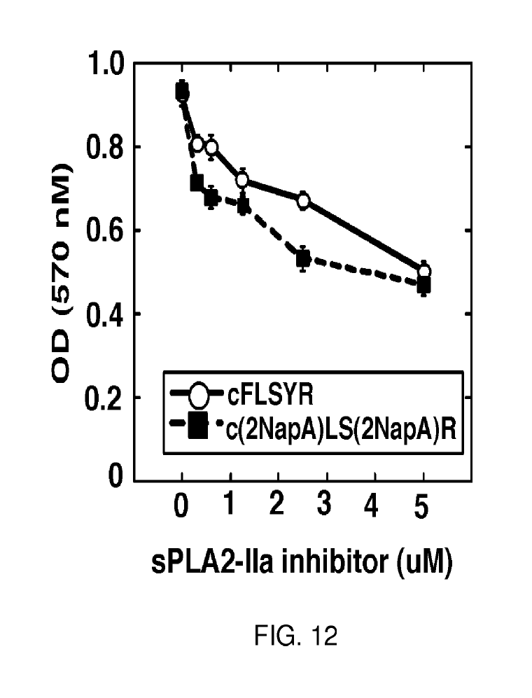

FIG. 12 is a graph of LNCaP cells cultured in 10% stripped medium in the presence of cFLSYR (μM) or c(2NapA)LS(2NapA)R (μM) for about 4 days with respect to optical density (570 nM);

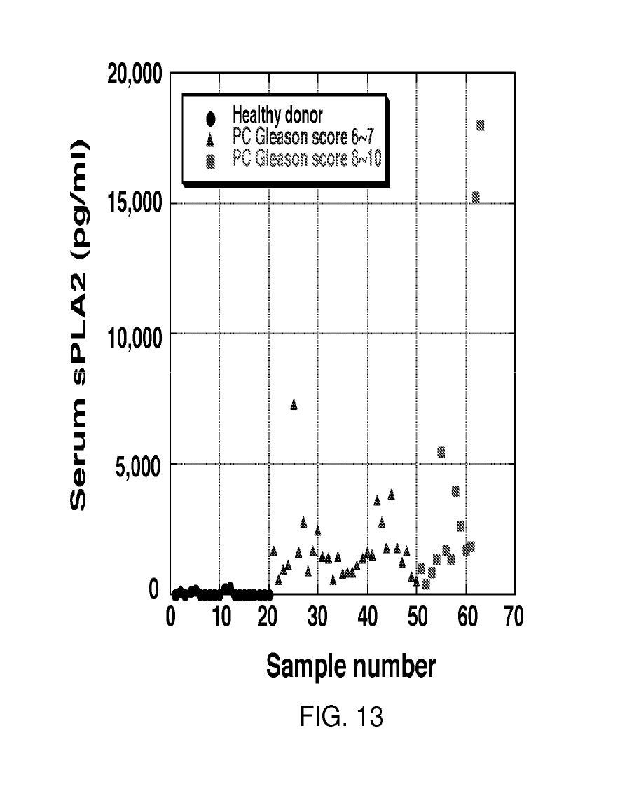

FIG. 13 is a graph of plasma samples from healthy donors (20 samples) and prostate cancer patients (43 samples) with respect to the level of serum sPLA2-IIA (pg/mL);

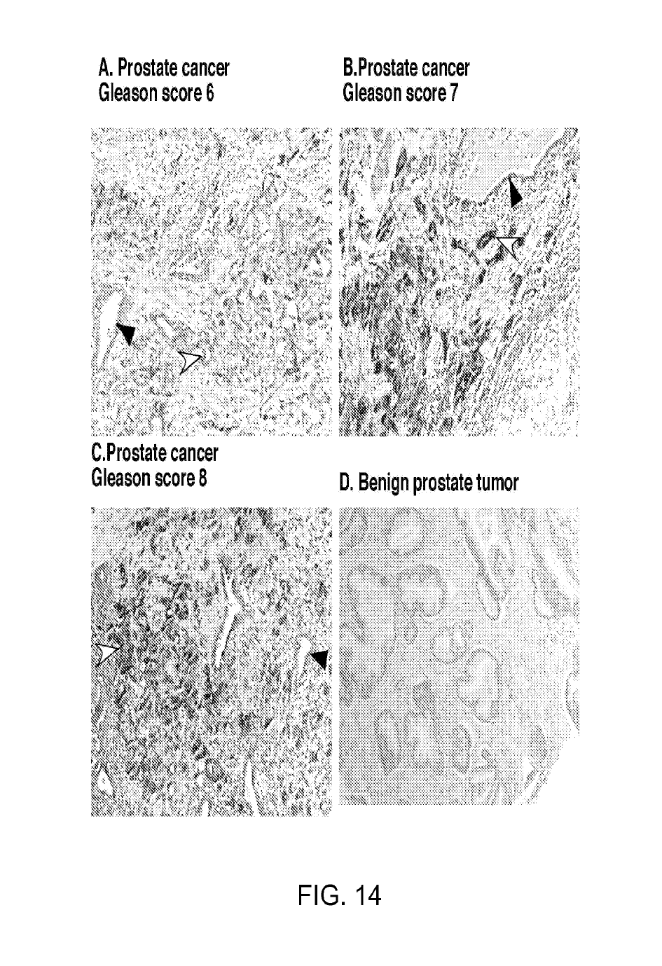

FIG. 14 is an immunohistochemistry stain of a lesion of Gleason score 6 (A), a lesion of Gleason score 7 (B), a lesion of Gleason score 8 (C), and benign prostate hyperplasia (D), wherein solid arrows indicate benign prostatic glands which are negative and serve as controls and open arrows indicate prostate cancer cells;

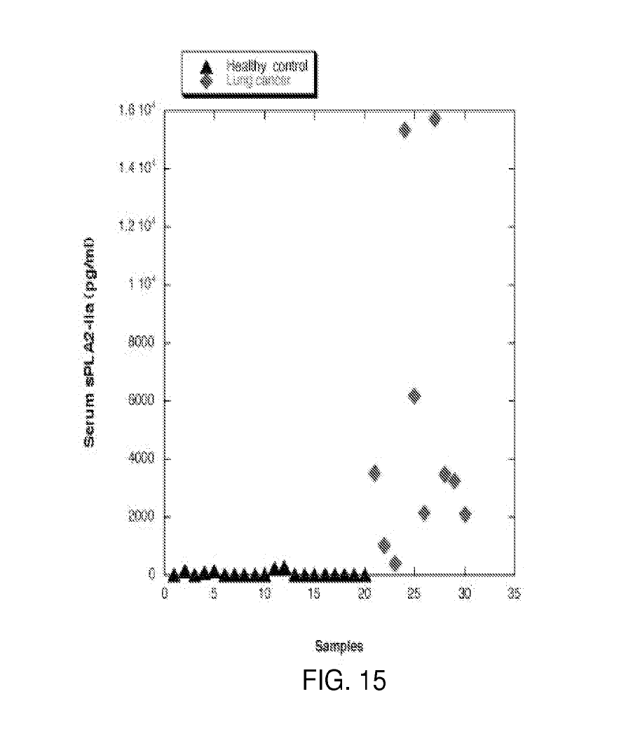

FIG. 15 is a graph of plasma samples from healthy donors (20 samples) and lung cancer patients (10 samples) with respect to the level of serum sPLA2-IIA (pg/mL);

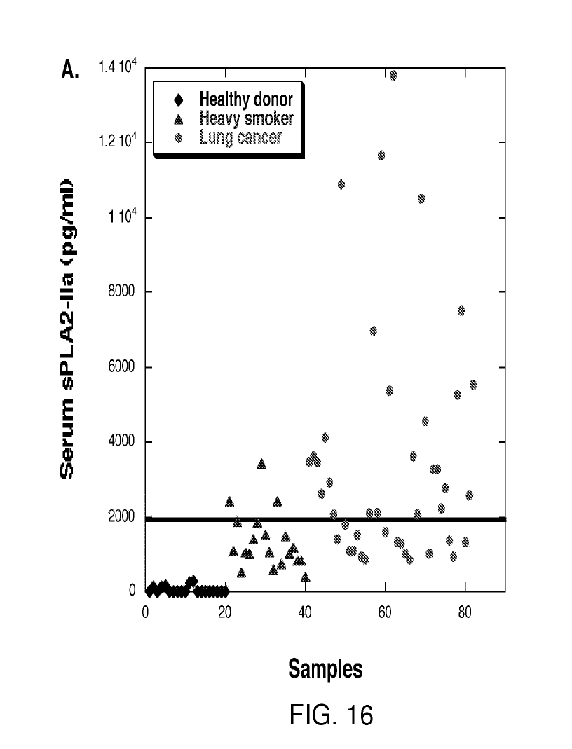

FIG. 16 is a graph of plasma samples from healthy donors, heavy smokers, and lung cancer patients with respect to the level of serum sPLA2-IIA (pg/mL);

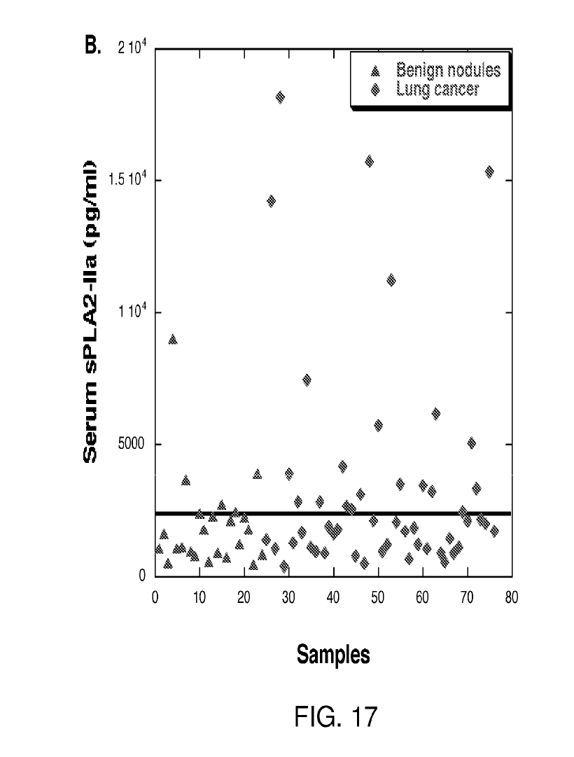

FIG. 17 is a graph of plasma samples from benign nodules and lung cancer patients with respect to the level of serum sPLA2-IIA (pg/mL);

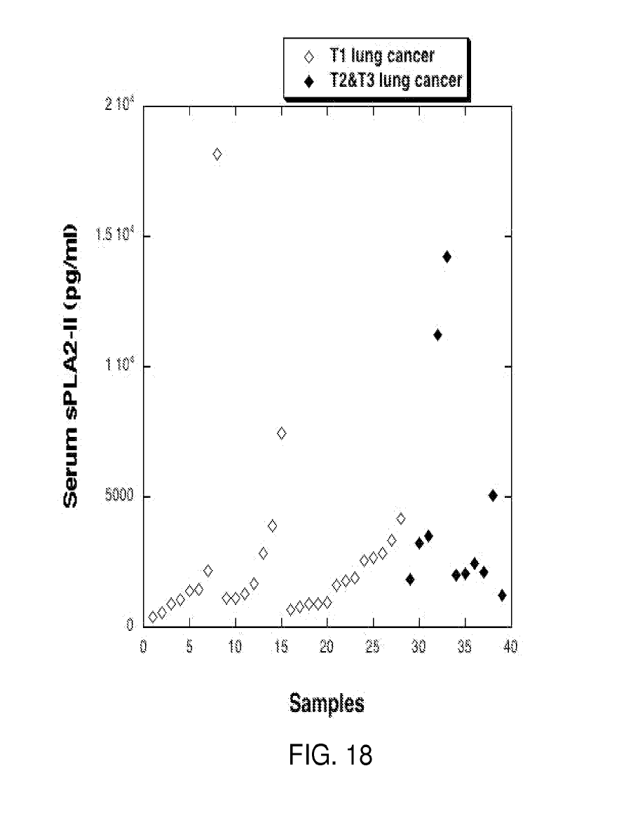

FIG. 18 is a graph of plasma samples from lung cancer patients with stage two and stage three cancer relative to early stage one cancer with respect to the level of sPLA2-IIA (pg/mL);

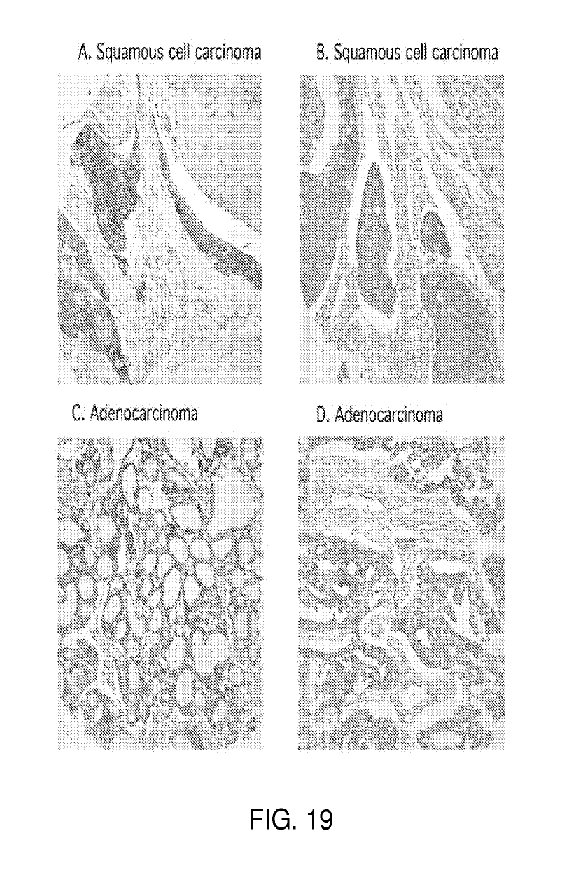

FIG. 19 is an immunohistochemistry stain of serum sPLA2-IIA expression in lung cancer specimens in squamous cell carcinoma (A and B) and adenocarcinoma (C and D); and

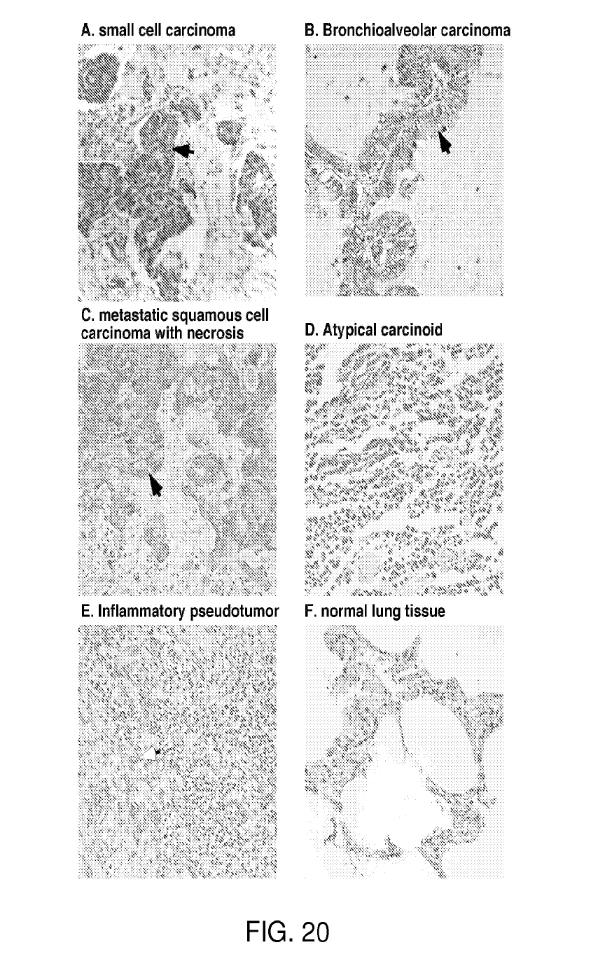

FIG. 20 is an immunhistochemistry stain of serum sPLA2-IIA expression in lung cancer specimens in small cell carcinoma (A), bronchioalveolar carcinoma (B), metastic squamous cell carcinoma (C), atypical carcinoid (D), inflammatory pseudo tumor (E), and normal lung tissue (F).

Skilled artisans appreciate that elements in the figures are illustrated for simplicity and clarity and are not necessarily drawn to scale. For example, the dimensions of some of the elements in the figures may be exaggerated relative to other elements, as well as conventional parts removed, to help to improve understanding of the various embodiments of the present invention.

The following terms are used in the present application:

As used herein, the terms “diagnosing”, “diagnosed”, and “diagnose” refer to determining the presence and/or absence of a disease or condition based upon an evaluation of physical signs, symptoms, history, laboratory test results, and/or procedures. Specifically, in the context of prostate cancer and/or lung cancer, diagnosing refers to determining the presence or absence of a disease or condition based upon an evaluation of the level of serum sPLA2-IIA.

As used herein, the term “positive diagnosis” refers to a determination of the presence of a disease or condition based upon an evaluation of physical signs, symptoms, history, laboratory test results, and/or procedures. In the context of prostate cancer, a positive diagnosis refers to a determination of the presence of prostate cancer based upon an evaluation of the level of serum sPLA2-IIA. Similarly, in the context of lung cancer, a positive diagnosis refers to a determination of the presence of lung cancer based upon an evaluation of the level of serum sPLA2-IIA.

As used herein, the term “negative diagnosis” refers to a determination of the absence of a disease or condition based upon an evaluation of physical signs, symptoms, history, laboratory test results, and/or procedures. In the context of prostate cancer, a negative diagnosis refers to a determination of the absence of prostate cancer based upon an evaluation of the level of serum sPLA2-IIA. Similarly, in the context of lung cancer, a negative diagnosis refers to a determination of the absence of lung cancer based upon an evaluation of the level of serum sPLA2-IIA.

In the context of serum sPLA2-IIA, the term “elevated level” refers to the level of serum sPLA2-IIA in a biological sample which is greater than a baseline level of serum sPLA2-IIA. For example, in the context of prostate cancer, an elevated level of serum sPLA2-IIA in blood plasma is from about 400 pg/mL to about 18,000 pg/mL, or from about 400 pg/mL to about 7,300 pg/mL, or from about 500 pg/mL to about 7,300 pg/mL, or from about 1,100 pg/mL to about 18,000 pg/mL. In the context of lung cancer, an elevated level of serum sPLA2-IIA in blood plasma is from about 400 pg/mL to about 16,000 pg/mL, or from about 400 pg/mL to about 7,500 pg/mL, or from about 1,200 pg/mL to about 15,000 pg/mL. In one embodiment, the elevation of the level of serum sPLA2-IIA in the biological sample is statistically significant.

Similarly, in the context of prostate specific antigen, the term “elevated level” refers to the level of prostate specific antigen in a biological sample which is greater than a baseline level of prostate specific antigen. For example, an elevated level of prostate specific antigen is from about 4 ng/mL to about 1,600 ng/mL.

As used herein, the term “baseline level” refers to the level of serum sPLA2-IIA in a biological sample from a subject who is not suffering from prostate cancer and/or lung cancer. In the context of prostate cancer, baseline level refers to the level of serum sPLA2-IIA in subjects with normal prostate tissue and/or in subjects with benign prostate disease. For example, in the context of prostate cancer, a baseline level of serum secretory sPLA2-IIA in blood plasma is from about 0 pg/mL to about 1,000 pg/mL. In the context of lung cancer, baseline level refers to the level of serum sPLA2-IIA in subjects with normal lung tissue, in subjects with benign lung diseases, and/or in subjects with benign solitary pulmonary nodules. For example, in the context of lung cancer, a baseline level of serum sPLA2-IIA in blood plasma is from about 0 pg/mL to about 2,000 pg/mL.

Similarly, in the context of prostate specific antigen, the term “baseline level” refers to the level of prostate specific antigen in a biological sample from a subject who is not suffering from prostate cancer. For example, a baseline level of prostate specific antigen is from about 0 ng/mL to about 4 ng/mL.

As used herein, the term “Gleason score” refers to system for scoring and/or measuring the aggressiveness of prostate cancer determined from tissue samples taken during a biopsy. A Gleason score may be used to help evaluate the prognosis of men with prostate cancer. Gleason scores range from about 6 to about 10. Generally, prostate cancers with higher Gleason scores are more aggressive.

As used herein, the term “cutoff value” refers to a threshold value which distinguishes subjects suffering from a disease or condition from subjects who are not suffering from the disease or condition. In the context of prostate cancer and lung cancer, an elevated level of serum sPLA2-IIA is greater than the cutoff value and a non-elevated level of serum sPLA2-IIA is less than the cutoff value. Specifically regarding prostate cancer, the cutoff value of serum sPLA2-IIA is about 1 ng/mL. Specifically regarding lung cancer, the cutoff value of serum sPLA2-IIA is about 2 ng/mL.

Embodiments of the present disclosure relate to methods for diagnosing prostate cancer and lung cancer in a subject. In one embodiment, a method for diagnosing prostate cancer in a subject is disclosed. In one particular embodiment, a method for diagnosing prostate cancer in a subject is disclosed, wherein the method comprises: (a) obtaining a biological sample from the subject; (b) determining a level of serum sPLA2-IIA in the biological sample; (c) comparing the level of serum sPLA2-IIA determined in step (b) with a baseline level of serum sPLA2-IIA; and (d) diagnosing prostate cancer in the subject, wherein an elevated level of serum sPLA2-IIA as compared to the baseline level correlates to a positive diagnosis of prostate cancer in the subject.

In one embodiment, the method for diagnosing prostate cancer in the subject comprises obtaining the biological sample from the subject in step (a). In one particular embodiment, the biological sample is blood. In a further embodiment, the biological sample is at least one of plasma and/or serum. In still a further embodiment, the biological sample is plasma. In another embodiment, the subject is human. Accordingly, obtaining the biological sample from the subject in step (a) of the method for diagnosing prostate cancer may comprise blood testing. In contrast to biopsies, blood testing is minimally invasive. Blood testing may be performed according to any blood testing methods known in the field. For example, in one particular embodiment, blood testing may be performed by extracting blood from the subject with a needle via venipuncture.

In yet another embodiment, the method for diagnosing prostate cancer in the subject comprises determining a level of serum sPLA2-IIA in the biological sample in step (b). In one embodiment, determining the level of serum sPLA2-IIA in the biological sample comprises performing an in vitro assay. The in vitro assay may be selected from the group consisting of immunoassays, aptamer-based assays, histological assays, cytological assays, and mRNA expression level assays. The in vitro assay should not be limited to those disclosed herein, however, but may be performed according to any methods known in the fields of biochemistry, molecular biology, and/or medical diagnostics. In one particular embodiment, the in vitro assay is an immunoassay comprising enzyme-linked immunosorbent assay.

In another embodiment, the method for diagnosing prostate cancer in the subject comprising comparing the level of serum sPLA2-IIA previously determined with a baseline level of serum sPLA2-IIA in step (c). The baseline level of serum sPLA2-IIA in step (c) may be determined in subjects with normal prostate tissue and/or in subjects with benign prostate disease. In one particular embodiment, the baseline level of serum sPLA2-IIA comprises a control for the method.

In yet another embodiment, the method for diagnosing prostate cancer in the subject comprises diagnosing prostate cancer in the subject in step (d), wherein an elevated level of serum sPLA2-IIA as compared to the baseline level correlates to a positive diagnosis of prostate cancer in the subject. In one particular embodiment, the elevated level of serum sPLA2-IIA is from about 400 pg/mL to about 18,000 pg/mL. In this particular embodiment, the biological sample has a Gleason score of from about 6 to about 10. In a further embodiment, the biological sample has a Gleason score of from about 8 to about 10. In another embodiment, the elevated level of serum sPLA2-IIA is from about 400 pg/mL to about 7,300 pg/mL. In this particular embodiment, the biological sample has a Gleason score of from about 6 to about 7.

In one particular embodiment, the elevated level of serum sPLA2-IIA correlates to a positive diagnosis of prostate cancer in the subject independent of the level of prostate specific antigen in the biological sample. Alternatively, in another embodiment, the elevated level of serum sPLA2-IIA correlates to a positive diagnosis of prostate cancer in the subject in conjunction with the level of prostate specific antigen in the sample. In this particular embodiment, the method for diagnosing prostate cancer in the subject further comprises determining a level of prostate specific antigen in the biological sample and comparing the level of prostate specific antigen with a baseline level of prostate specific antigen. In this embodiment, an elevated level of prostate specific antigen as compared to the baseline level correlates to a positive diagnosis of prostate cancer. In one embodiment, the level of prostate specific antigen is determined via in vitro assay as previously described above. In one particular embodiment, the in vitro assay is an immunoassay comprising enzyme-linked immunosorbent assay.

In another embodiment, the elevated level of serum sPLA2-IIA increases with the progression (i.e. increasing severity) of the prostate cancer. Prostate cancer increases in severity in the order of stages. For example, in stage two prostate cancer, the elevated level of serum sPLA2-IIA in the biological sample is from about 500 pg/mL to about 7,300 pg/mL. Additionally, as another example, in stage three prostate cancer, the elevated level of serum sPLA2-IIA in the biological sample is from about 1,100 pg/mL to about 18,000 pg/mL.

Accordingly, in one embodiment, the method for diagnosing prostate cancer in the subject further comprises determining a stage of the prostate cancer, wherein an elevated level of serum sPLA2-IIA in the biological sample of from about 500 pg/mL to about 7,300 pg/mL correlates to a diagnosis of stage two prostate cancer in the subject. In another embodiment, an elevated level of serum sPLA2-IIA in the biological sample of from about 1,100 pg/mL to about 18,000 pg/mL correlates to a diagnosis of stage three prostate cancer in the subject.

In still another embodiment, a non-elevated level of serum sPLA2-IIA in the biological sample of serum sPLA2-IIA as compared to the baseline level correlates to a negative diagnosis of prostate cancer in the subject. In one particular embodiment, the non-elevated level of serum sPLA2-IIA as compared to the baseline level correlates to a diagnosis of normal prostate tissue and/or benign prostatic diseases. In a further embodiment, the benign prostatic disease comprises benign prostatic hyperplasia.

In yet another embodiment, the method for diagnosing prostate cancer in the subject further comprises determining a cutoff value, wherein the non-elevated level of serum sPLA2-IIA is less than the cutoff value. In one particular embodiment, the cutoff value is about 1 ng/mL.

In another embodiment, a method for diagnosing lung cancer in a subject is disclosed, wherein the method comprises: (a) obtaining a biological sample from the subject; (b) determining a level of serum sPLA2-IIA in the biological sample; (c) comparing the level of serum sPLA2-IIA determined in step (b) with a baseline level of serum sPLA2-IIA; and (d) diagnosing lung cancer in the subject, wherein an elevated level of serum sPLA2-IIA as compared to the baseline level correlates to a positive diagnosis of lung cancer in the subject.

In one embodiment, the method for diagnosing lung cancer in the subject comprises obtaining the biological sample from the subject in step (a). In one particular embodiment, the biological sample is blood. In a further embodiment, the biological sample is at least one of plasma and/or serum. In still a further embodiment, the biological sample is plasma. In another embodiment, the subject is human. Accordingly, obtaining the biological sample from the subject in step (a) of the method for diagnosing lung cancer may comprise blood testing as previously described above.

In another embodiment, the method for diagnosing lung cancer in the subject comprises determining a level of serum sPLA2-IIA in the biological sample in step (b). In one embodiment, determining the level of serum sPLA2-IIA in the biological sample comprises performing an in vitro assay. The in vitro assay may be performed as previously described above. In one particular embodiment, the in vitro assay is an immunoassay comprising enzyme-linked immunosorbent assay.

In another embodiment, the method for diagnosing lung cancer in the subject comprising comparing the level of serum sPLA2-IIA previously determined with a baseline level of serum sPLA2-IIA in step (c). The baseline level of serum sPLA2-IIA in step (c) may be determined in subjects with normal lung tissue and/or in subjects with benign lung diseases. In one particular embodiment, the baseline level of serum sPLA2-IIA comprises a control for the method.

In yet another embodiment, the method for diagnosing lung cancer in the subject comprises diagnosing lung cancer in the subject in step (d), wherein an elevated level of serum sPLA2-IIA as compared to the baseline level correlates to a positive diagnosis of lung cancer in the subject. In one particular embodiment, the elevated level of serum sPLA2-IIA is from about 400 pg/mL to about 16,000 pg/mL. In this particular embodiment, the elevated level of sPLA2-IIA correlates to a positive diagnosis of lung cancer. The lung cancer is selected from the group consisting of non-small cell lung cancer, small cell carcinoma, and metastatic squamous cell carcinoma. In a further aspect, the non-small cell lung cancer is selected from the group consisting of squamous cell carcinoma, adenocarcinoma, and bronchioalveolar carcinoma.

In another embodiment, the elevated level of serum sPLA2-IIA increases with the progression (i.e. increasing severity) of the lung cancer. Lung cancer increases in severity in the order of stages. For example, in stage one lung cancer, the elevated level of serum sPLA2-IIA in the biological sample is from about 400 pg/mL to about 7,500 pg/mL. Additionally, as another example, in stage two or stage three lung cancer, the elevated level of serum sPLA2-IIA in the biological sample is from about 1,200 pg/mL to about 15,000 pg/mL.

Accordingly, in one embodiment, the method for diagnosing lung cancer in the subject further comprises determining a stage of the lung cancer, wherein an elevated level of serum sPLA2-IIA in the biological sample of from about 400 pg/mL to about 7,500 pg/mL correlates to a diagnosis of stage one lung cancer in the subject. In another embodiment, an elevated level of serum sPLA2-IIA in the biological sample of from about 1,200 pg/mL to about 15,000 pg/mL correlates to a diagnosis of stage two or stage three lung cancer in the subject.

In still another embodiment, a non-elevated level of serum sPLA2-IIA in the biological sample of serum sPLA2-IIA as compared to the baseline level correlates to a negative diagnosis of lung cancer in the subject. In one particular embodiment, the non-elevated level of serum sPLA2-IIA as compared to the baseline level correlates to a diagnosis of normal lung tissue, benign lung diseases, and/or benign solitary pulmonary nodules. In a further embodiment, the benign solitary pulmonary nodules comprise inflammatory pseudo tumors.

In yet another embodiment, the method for diagnosing lung cancer in the subject further comprises determining a cutoff value, wherein the non-elevated level of serum sPLA2-IIA is less than the cutoff value. In one particular embodiment, the cutoff value is about 2 ng/mL.

### EXAMPLES

The following non-limiting examples illustrate the methods of the present disclosure.

### Example 1

### Regulation of Human sPLA2-IIA Gene Expression Mediated by the EGFR/HER2-Elicited Pathways

Experimental Protocol. The role of sPLA2-IIA gene regulation in prostate cancer cells via the HER/HER2-PI3K-Akt-NF-κB pathway was investigated. A reporter assay was performed by transiently transfecting sPLA2-IIA(-800)-Luc (˜0.25 μg/well) reporter in LNCaP-AI cells (˜10⁵ cells/well in 12-well plate). The cells were then treated with epidermal growth factor (hereinafter “EGF”) (˜100 ng/mL) without or with EGFR inhibitors Erlotinib (˜20 μM) and Gefitinib (˜20 μM), EGFR/HER2 dual inhibitors Lapatinib (˜20 μM) and CI-1033 (˜8 μM), phosphoinositide 3-kinase (hereinafter “PI3K”) inhibitor LY294002 (˜20 μM), and NF-κB inhibitor Bortezomib (˜20 μM) for about 24 hours. A luciferase assay was performed according to a standard protocol with Renilla luciferase as an internal control.

Another reporter assay was performed wherein LNCaP-AI cells were starved in 1% stripped medium for about 24 hours. The cells were the treated with Erlotinib (˜20 μM), Gefitinib (˜20 μM) Lapatinib (˜20 μM), CI-1033 (˜8 μM), LY294002 (˜20 μM), Bortezomib (˜20 μM) and/or Heregulin-α (˜50 ng/mL) without or with EGF (˜100 ng/mL) for about 24 hours. Cell extracts were then prepared and subjected to western blot analysis for sPLA2-IIA, P-Akt, Akt, and β-actin.

Finally, LNCaP-AI cells were starved in 1% stripped medium for about 24 hours. The cells were then treated with Erlotinib (˜20 μM), Gefitinib (˜20 μM), Lapatinib (˜20 μM), CI-1033 (˜8 μM), LY294002 (˜20 μM), and Bortezomib (˜20 μM) for about 24 hours. Cell culture medium was collected from each sample and subjected to ELISA for sPLA2-IIA. The condition medium samples were diluted 10 times for ELISA. The average of duplicate samples was converted to nanogram per milliliter against standard curve. The data represent one of five repeated experiments.

Experimental Results. As shown in FIG. 1, EGF significantly stimulated the promoter activity of sPLA2-IIA gene, which was blocked by EGFR inhibitors Erlotinib and Gefitinib, EGFR/HER2 dual inhibitors Lapatinib and CI-1033, PI3K inhibitor LY294002, and NF-κB inhibitor Bortezomib. This data indicates that the elevated signaling of the HER/HER2-P13K-Akt-NF-κB pathway upregulates expression of sPLA2-IIA gene at the transcriptional level. Data are presented as the mean (±SD) of duplicative values of a representative experiment that was independently repeated for five times.

As shown in FIGS. 2 and 3, EGF stimulated sPLA2-IIA expression. Moreover, as shown in FIGS. 2-4, among the inhibitors examined, Lapatinib, LY294002 and Bortezomib dramatically downregulated sPLA2-IIA protein expression in both basal states and in the setting of EGF-induced expression, whereas Erlotinib, Gefitinib, and CI-1033 had a moderate impact on sPLA2-IIA protein expression. As shown in FIG. 5, sPLA2-IIA was also expressed in LAPC-4 and DU145 cells, but not PC-3 cells, which were inhibited by Lapatinib via blocking PI3K-Akt signaling. Finally, as shown in FIG. 6, HER3 ligand Heregulin-α enhanced Akt phosphorylation and sPLA2-IIA expression via PI3K-Akt signaling in LNCaP cells.

As shown in FIG. 7, Lapatinib, LY294002, and Bortezomib significantly inhibited sPLA2-IIA secretion, whereas Erlotinib, Gefitinib and CI-1033 had a moderate effect in LNCaP-AI cells.

### Example 2

### SPLA2-IIA Gene is Overexpressed in Androgen-Independent LNCaP-AI Cells

Experimental Protocol. Expression levels of sixteen thousand genes in LNCaP-AI cells, an androgen-independent cell line, and LNCaP cells, an androgen-dependent cell line, were compared using DNA oligonucleotide microarray analysis. The androgen-independent cell line was developed from its parental androgen-dependent cell line.

The expression levels of sPLA2-IIA in LNCaP-AI cells and LNCaP cells were determined by real-time RT-PCR analysis at the mRNA level and by western blot analysis at the protein level. Additionally, quantitative analyses of the level of sPLA2-IIA in LNCaP-AI cells and LNCaP cells was performed by ELISA assay. More specifically, the LNCaP-AI cells and LNCaP cells (˜500,000 cells/well in 6 well plate) were cultured in stripped medium for about 2 days. The medium samples were then collected and subjected to ELISA analysis using human sPLA2 type IIa EIA kit, Catalog No. 585000 (Cayman Chemical Company, Ann Arbor, Mich.).

Experimental Results. As shown in Table 1 below, sPLA2-IIA, Vav3, and p21/WAF were overexpressed in the LNCaP-AI cell line. Overexpression of these genes in the LNCaP-AI cell line implicates that elevated activities of these genes support androgen-independent growth in prostate cancer cells.

TABLE 1

| | LNCaP | Spot | Spot | AI vs. | Spot | Spot | | UniGene ID/ |
|---|---|---|---|---|---|---|---|---|
| Name | vs. AI | Norm | Norm | LNCaP | Norm | Norm | Comments | Gene Symbol |
| H011554 | 8.8 | 4 | 1169 | 8.4 | 1300 | 5 | Vav 3 oncogene | 267659/ |
| | | | | | | | | VAV3 |
| H002863 | 5.5 | 348 | 10935 | 4.5 | 12971 | 282 | sPLA2-IIA | 76422/ |
| | | | | | | | | PLA2G2A |
| H002239 | 4.3 | 452 | 5858 | 2.2 | 4285 | 490 | Cyclin-dependent | 179665/ |
| | | | | | | | kinase inhibitor | CDKN1A |
| | | | | | | | 1A (p21, Clp1 | |
| H002149 | 4.3 | 1028 | 13556 | 2.0 | 14941 | 1755 | Cyclin-dependent | 179665/ |
| | | | | | | | kinase inhibitor | CDKN1A |
| | | | | | | | 1A (p21, Clp1 | |

As shown in FIGS. 8 and 9, sPLA2-IIA overexpression in LNCaP-AI cells was respectively confirmed by real-time RT-PCR analysis and western blot analysis. Additionally, as shown in FIG. 10, LNCaP-AI cells secrete a greater amount of sPLA2-IIA into the medium as compared to the amount of sPLA2-IIA secreted by LNCaP into the medium.

### Example 3

### Role of sPLA2-IIA on Growth of Prostate Cancer Cells

Experimental Protocol. The role of sPLA2-IIA in the growth of prostate cancer cells was studied by MTT assay. Specifically, LNCaP-AI cells were cultured in 10% stripped medium in the presence of EGF (ng/mL) or sPLA2-IIA (ng/mL) for about 4 days, followed by performance of an MTT assay. The role of sPLA2-IIA on prostate cancer cell growth was also studied by blocking the activity of sPLA2-IIA in LNCaP-AI cells. The activity of sPLA2-IIA was blocked in LNCaP-AI cells by administering the peptide inhibitor cFLYSYR (μM) or c(NapA)R (μM) for about 4 days, followed by performance of an MTT assay.

Experimental Results. As shown in FIG. 11, sPLA2-IIA functions as a growth factor and stimulates prostate cancer cell growth in an androgen independent manner in LNCaP-AI cells. As shown in FIG. 12, blocking the activity of sPLA2-IIA in LNCaP-AI cells significantly inhibited prostate cancer cell growth. Thus, these data confirm that sPLA2-IIA plays a role in the growth of prostate cancer cells and also indicate that sPLA2-IIA overexpression contributes to prostate cancer tumorigenesis and progression.

### Example 4

### Serum sPLA2-IIA is Elevated in Prostate Cancer Patients

Experimental Protocol. A series of studies were conducted in which serum sPLA2-IIA levels were examined in prostate cancer patients in comparison to healthy donors. In one study, (hereinafter “the 43 study”), 43 plasma samples from prostate cancer patients were obtained from the University of Cincinnati Cancer Center Tumor Bank (Cincinnati, Ohio). Of the 43 plasma samples from prostate cancer patients, 13 plasma samples had Gleason scores of about 8 to about 10 and 30 plasma samples had Gleason scores of about 6 to about 7. Additionally, 20 plasma samples from healthy donors were obtained from the Cincinnati Hoxworth Blood Center (Cincinnati, Ohio). All plasma samples were diluted ten times and then subjected to duplicate ELISA analysis using the kit previously discussed. The average of the duplicate sample was calculated to present as pg/mL based on the standard curve of each experiment.

Immunohistochemistry analysis of sPLA2-IIA expression in prostate cancer and benign prostatic hyperplasia (hereinafter “BPH”) specimens was also performed. BPH specimens served as a control. Prostate cancer and BPH specimens were obtained as a prostate disease spectrum tissue array (Biomatrix US, Rockville, Md.) and the University of Cincinnati Cancer Center Tumor Bank.

Experimental Results. As shown in FIG. 13, in the 43 study, all prostate cancer patients showed an elevated level of serum sPLA2-IIA as compared to the healthy donors. The levels of sPLA2-IIA in prostate cancer patients ranged from ˜400 pg/mL to ˜18,000 pg/mL. Additionally, sPLA2-IIA was not detected in 15 of the plasma samples from the healthy donors and was less than ˜275 pg/mL in 5 of the plasma samples from the healthy donors. The sPLA2-IIA levels were correlated significantly with the presence of prostate cancer (p=0.0024, unpaired t test). Moreover, among the 20 healthy donors, 6 were greater than 60 years of age and 4 were greater than 50 years of age indicating that age was not a contributing factor for higher sPLA2-IIA levels in samples from cancer patients.

Also shown in FIG. 13, further analysis revealed that sPLA2-IIA levels were significantly higher in samples from prostate cancers with high Gleason score (˜8 to ˜10) than those with intermediate Gleason score (˜6 to ˜7) (p=0.0252, unpaired t test). Moreover, as shown in Table 2 below, sPLA2-IIA levels were correlated with the pathological stages of prostate cancer. Specifically, the levels in samples from patients with T3 cancer were significantly higher than those with T2 cancer (p=0.0298, nonparametric test). These results were confirmed by association analysis using the optimum cutoff value of serum sPLA2-IIA (˜1.0 ng/mL) determined by ROC curve analysis. The two samples with the highest serum sPLA2 levels (˜15,209 pg/mL and ˜18,003 pg/mL) originated from patients with stage T3 disease in prostate cancers with high Gleason scores of ˜9 and ˜10.

TABLE 2

| PC stage | <1000 pg/ml | >1000 pg/ml | Total case # |
|---|---|---|---|
| T2 | 6 (35%) | 11 (65%) | 17 |
| T3 | 0 | 16 (100%) | 16 |

Fisher's exact test: P = 0.01

As shown in FIG. 14, immunohistochemistry staining also demonstrated elevated expression levels of sPLA2-IIA in tumor specimens with high Gleason scores and advanced cancer stage. Moreover, none of BPH specimens examined were significantly positive for sPLA2-IIA staining. As shown in (A) of FIG. 14, moderate cytyoplasmic granular staining was demonstrated in a lesion of Gleason score of ˜6. As shown in (B) of FIG. 14, a strong and diffuse cytoplasmic granular staining was demonstrated in a lesion of Gleason score of ˜7. Additionally, as shown in (C) of FIG. 14, the strongest staining was demonstrated in a lesion of Gleason score of ˜8. Moreover, as shown in (D) of FIG. 14, BPH specimens were negative for sPLA2-IIA staining.

### Example 5

### Serum sPLA2-IIA is Elevated in Lung Cancer Patients

Experimental Protocol. Serum sPLA2-IIA levels were examined in lung cancer patients in comparison with healthy donors. Specifically, 10 plasma samples from lung cancer patients were obtained from the University of Cincinnati Cancer Center Tumor Bank. 20 plasma samples from healthy donors were obtained from the Cincinnati Hoxworth Blood Center. All plasma samples were diluted ten times and then subjected to duplicate ELISA analysis using the kit previously discussed. The average of the duplicate sample was calculated to present as pg/mL based on the standard curve of each experiment.

Serum sPLA2-IIA levels were also examined in lung cancer patients in comparison with patients with benign nodules and plasma samples were obtained from the University of Cincinnati Cancer Center Tumor Bank. Additionally, serum sPLA2-IIA levels were also examined in lung cancer patients in comparison with healthy donors and heavy smokers without lung cancer and plasma samples were obtained from the “Genetic Epidemiology of Lung Cancer” Project, a family lung cohort study. Plasma samples from healthy donors were obtained form the Cincinnati Hoxworth Blood Center. All plasma samples were diluted ten times and then subjected to duplicate ELISA analysis using the kit previously discussed. The concentration of sPLA2-IIA in plasma was tested in duplicate and determined against a standard curve for each ELISA assay.

Immnohistochemistry analysis of sPLA2-IIA expression in an array of lung tissue specimens was also performed. Specifically, immunohistochemistry analysis of sPLA2-IIA expression was performed in the following lung tissue specimens: squamous cell carcinoma specimens, adenocarcinoma specimens, bronchioalveolar carcinoma specimens, small cell carcinoma specimens, metastatic squamous cell carcinoma specimens, atypical carcinoid (malignant tumor) specimens, inflammatory pseudo tumor specimens, and normal lung tissue specimens. Lung tissue specimens were obtained as a lung disease spectrum tissue array (Biomatrix US).

Experimental Results. As shown in FIG. 15, lung cancer patients showed significantly elevated levels of serum sPLA2-IIA as compared to the healthy donors (P<0.001) analyzed by one-way Analysis of Variance (hereinafter “ANOVA”). Additionally, sPLA2-IIA was not detected in 15 of the plasma samples from the healthy donors and was less than ˜275 pg/mL in 5 of the plasma samples from the healthy donors. Moreover, among the 20 healthy donors, 6 were greater than 60 years of age and 4 were greater than 50 years of age indicating that age was not a contributing factor for higher sPLA2-IIA levels in samples from lung cancer patients.

As shown in FIG. 16, the levels of serum sPLA2-IIA in plasma samples in lung cancer patients were significantly higher than those in heavy smokers without lung cancers (P=0.0002). Specifically, the levels of serum sPLA2-IIA ranged from about 0 pg/mL to about 245 pg/mL in healthy donors and age was not significantly associated with the levels of serum sPLA2-IIA in the normal cohort. ROC analysis resulted in 93% positive predictive value (hereinafter “PPV”) with a cutoff value of about 1.9 ng/mL. Moreover, as shown in Table 3 below, specificity and sensitivity of heavy smoker plasma samples and lung cancer plasma samples were 85% and 62% respectively.

TABLE 3
Serum sPLA2-II

| | | |
|---|---|---|
| <1.9 ng/ml | >1.9 ng/ml | Total case# |

Cutoff value = 1.9 ng/ml
PPV = 93%
Specificity = 85%
Sensitivity = 62%

As shown in FIG. 17, the levels of serum sPLA2-IIA in plasma samples in lung cancer patients were significantly higher than those in patients with benign lung nodules (unpaired t test: P=0.028). ROC analysis resulted in 80% PPV with a cutoff value of 2.36 ng/mL. Moreover, as shown in Table 4 below, specificity and sensitivity of the benign nodule plasma samples and lung cancer plasma samples were 75% and 40%.

TABLE 4
Serum sPLA2-IIa

| | <2.36 ng/ml | >2.36 ng/ml | Total case# |
|---|---|---|---|
| Benign nodule | 18 | 6 | 24 |
| Lung cancer | 31 | 21 | 52 |

Cutoff value = 2.36 ng/ml
PPV = 80%
Specificity = 75%
Sensitivity = 40%

As shown in FIG. 18, the levels of serum sPLA2-IIA were significantly associated with T2 and T3 stage relative to the early T1 stage of lung cancer.

As shown in FIGS. 19 and 20, among 100 lung biopsies examined, sPLA2-IIA was overexpressed in 100% of squamous cell carcinoma, 100% of adenocarcinoma, and 100% of bronchioalveolar carcinoma. sPLA2-IIA was also overexpressed in 70% of small cell carcinoma and in 90% of metastatic squamous cell carcinoma. Additionally, sPLA2-IIA was not detected in atypical carcinoid (malignant tumor), inflammatory pseudo tumor, and normal lung tissue. Moreover, IHC analysis demonstrated that moderate increased serum sPLA2-IIA in inflammation was due to expression of sPLA2-IIA by infiltrated macrophage and endothelial cells in new blood vessels of the inflammation site.

It is noted that terms like “preferably,” “generally,” “commonly,” and “typically” are not utilized herein to limit the scope of the claimed invention or to imply that certain features are critical, essential, or even important to the structure or function of the claimed invention. Rather, these terms are merely intended to highlight alternative or additional features that may or may not be utilized in a particular embodiment of the present invention.

For the purposes of describing and defining the present invention it is noted that the term “substantially” is utilized herein to represent the inherent degree of uncertainty that may be attributed to any quantitative comparison, value, measurement, or other representation. The term “substantially” is also utilized herein to represent the degree by which a quantitative representation may vary from a stated reference without resulting in a change in the basic function of the subject matter at issue.

All documents cited are incorporated herein by reference; the citation of any document is not to be construed as an admission that it is prior art with respect to the present invention.

While particular embodiments of the present invention have been illustrated and described, it would be obvious to one skilled in the art that various other changes and modifications can be made without departing from the spirit and scope of the invention. It is therefore intended to cover in the appended claims all such changes and modifications that are within the scope of this invention.

1. A method for diagnosing prostate cancer in a subject, the method comprising:
(a) obtaining a biological sample from the subject;
(b) determining a level of serum secretory phospholipase A₂-IIA in the biological sample;
(c) comparing the level of serum secretory phospholipase A₂-IIA determined in step (b) with a baseline level of serum secretory phospholipase A₂-IIA; and
(d) diagnosing prostate cancer in the subject, wherein an elevated level of serum secretory phospholipase A₂-IIA as compared to the baseline level correlates to a positive diagnosis of prostate cancer in the subject.

2. The method of claim 1, wherein the biological sample is blood.

3. The method of claim 2, wherein the biological sample is plasma.

4. The method of claim 1, wherein the subject is human.

5. The method of claim 1, wherein determining the level of serum secretory phospholipase A₂-IIA in the biological sample comprises performing an in vitro assay.

6. The method of claim 5, wherein the in vitro assay is selected from the group consisting of immunoassays, aptamer-based assays, histological assays, cytological assays, and mRNA expression level assays.

7. The method of claim 6, wherein the in vitro assay is an immunoassay comprising enzyme-linked immunosorbent assay.

8. The method of claim 1, wherein the elevated level of serum secretory phospholipase A₂-IIA is from about 400 pg/mL to about 18,000 pg/mL.

9. The method of claim 8, wherein the biological sample has a Gleason score of from about 6 to about 10.

10. The method of claim 9, wherein the biological sample has a Gleason score of from about 8 to about 10.

11. The method of claim 8, wherein the elevated level of serum secretory phospholipase A₂-IIA is from about 400 pg/mL to about 7,300 pg/mL.

12. The method of claim 11, wherein the biological sample has a Gleason score of from about 6 to about 7.

13. The method of claim 8, wherein the elevated level of serum secretory phospholipase A₂-IIA correlates to a positive diagnosis of prostate cancer in the subject independent of the level of prostate specific antigen in the biological sample.

14. The method of claim 8, further comprising determining a level of prostate specific antigen in the biological sample and comparing the level of prostate specific antigen with a baseline level of prostate specific antigen, wherein an elevated level of prostate specific antigen as compared to the baseline level correlates to a positive diagnosis of prostate cancer.

15. The method of claim 8, wherein the elevated level of serum secretory phospholipase A₂-IIA increases with progression of the prostate cancer.

16. The method of claim 8, further comprising determining a stage of the prostate cancer, wherein an elevated level of serum secretory phospholipase A₂-IIA in the biological sample of from about 500 pg/mL to about 7,300 pg/mL correlates to a diagnosis of stage two prostate cancer in the subject.

17. The method of claim 8, further comprising determining a stage of the prostate cancer, wherein an elevated level of serum secretory phospholipase A₂-IIA in the biological sample of from about 1,100 pg/mL to about 18,000 pg/mL correlates to a diagnosis of stage three prostate cancer in the subject.

18. The method of claim 1, wherein a non-elevated level of serum secretory phospholipase A₂-IIA as compared to the baseline level correlates to a negative diagnosis of prostate cancer in the subject.

19. The method of claim 18, wherein the non-elevated level of serum secretory phospholipase A₂-IIA correlates to a diagnosis of one or more of normal prostate tissue and benign prostatic diseases.

20. The method of claim 19, wherein benign prostatic disease is benign prostatic hyperplasia.

21. The method of claim 18, further comprising determining a cutoff value, wherein the non-elevated level of serum secretory phospholipase A₂-IIA is less than the cutoff value.

22. The method of claim 21, wherein the cutoff value is about 1 ng/mL.

23. A method for diagnosis of lung cancer in a subject, the method comprising:
(a) obtaining a biological sample from the subject;
(b) determining a level of serum secretory phospholipase A₂-IIA, in the biological sample;
(c) comparing the level of serum secretory phospholipase A₂-IIA determined in step (b) with a baseline level of serum secretory phospholipase A₂-IIA; and
(d) diagnosing lung cancer in the subject, wherein an elevated level of serum secretory phospholipase A₂-IIA as compared to the baseline level correlates to a positive diagnosis of lung cancer in the subject.

24. The method of claim 23, wherein the biological sample is blood.

25. The method of claim 24, wherein the biological sample is plasma.

26. The method of claim 23, wherein the subject is human.

27. The method of claim 23, wherein determining the level of serum secretory phospholipase A₂-IIA in the biological sample from the subject comprises performing an in vitro assay.

28. The method of claim 27, wherein the in vitro assay is selected from the group consisting of immunoassays, aptamer-based assays, histological assays, cytological assays, and mRNA expression level assays.

29. The method of claim 28, wherein the in vitro assay is an immunoassay comprising enzyme-linked immunosorbent assay.

30. The method of claim 23, wherein the elevated level of serum secretory phospholipase A₂-IIA is from about 400 pg/mL to about 16,000 pg/mL.

31. The method of claim 30, wherein the elevated level of serum secretory phospholipase A₂-IIA correlates to a positive diagnosis of lung cancer, wherein the lung cancer is selected from the group consisting of non-small cell lung cancer, small cell carcinoma, and metastatic squamous cell carcinoma.

32. The method of claim 31, wherein the non-small cell lung cancer is selected from the group consisting of squamous cell carcinoma, adenocarcinoma, and bronchioalveolar carcinoma.

33. The method of claim 30, wherein the elevated level of serum secretory phospholipase A₂-IIA increases with progression of the lung cancer.

34. The method of claim 30, further comprising determining a stage of the lung cancer, wherein an elevated level of serum secretory phospholipase A₂-IIA in the biological sample of from about 400 pg/mL to about 7,500 pg/mL correlates to a diagnosis of stage one lung cancer in the subject.

35. The method of claim 30, further comprising determining a stage of the lung cancer, wherein an elevated level of serum secretory phospholipase A₂-IIA in the biological sample of from about 1,200 pg/mL to about 15,000 pg/mL correlates to a diagnosis of stage two or stage three lung cancer in the subject.

36. The method of claim 23, wherein a non-elevated level of serum secretory phospholipase A₂-IIA as compared to the baseline level correlates to a negative diagnosis of lung cancer in the subject.

37. The method of claim 36, wherein the non-elevated level of serum secretory phospholipase A₂-IIA correlates to a diagnosis of one or more of normal lung tissue, benign lung diseases, and benign solitary pulmonary nodules.

38. The method of claim 37, wherein the benign solitary pulmonary nodules comprise inflammatory pseudo tumors.

39. The method of claim 36, further comprising determining a cutoff value, wherein the non-elevated level of serum secretory phospholipase A₂-IIA is less than the cutoff value.

40. The method of claim 39, wherein the cutoff value is about 2 ng/mL.
